(19) United States
(12) Patent Application Publication
(10) Pub. No.: US 2015/0329635 A1
(43) Pub. Date: Nov. 19, 2015

(54) SUPPRESSION OF LEPTIN ACTION FOR TREATMENT OF PULMONARY INFECTIONS

(71) Applicant: The University of Vermont and State Agricultural College, Burlington, VT (US)

(72) Inventor: Benjamin T. Suratt, Essex, VT (US)

(21) Appl. No.: 14/713,754

(22) Filed: May 15, 2015

Related U.S. Application Data
(60) Provisional application No. 61/993,845, filed on May 15, 2014.

(51) Int. Cl. C07K 16/26; G01N 33/74; A61K 39/395; A61K 9/00; A61K 45/06

(52) U.S. Cl. CPC C07K 16/26; A61K 9/0073; A61K 45/06; A61K 39/3955; G01N 33/74; C07K 2317/569; C07K 2317/76; G01N 2333/575; G01N 2800/12

(57) ABSTRACT

Provided is a method of assessing risk of developing pulmonary infection in an individual or a population comprising determining circulating leptin levels in the individuals and comparing the levels or normal controls, and if the leptin levels are higher than in the control, identifying the individual to be at risk of developing pulmonary infection. Also provided are methods of reducing the severity of, or preventing pulmonary infections in individuals with elevated leptin levels by administration of agents that that suppress the levels of leptin or interfere with its actions.

### CROSS-REFERENCE TO RELATED APPLICATIONS

This application claims priority to U.S. provisional application No. 61/993,845, filed on May 15, 2014, the disclosure of which is incorporated herein by reference.

### STATEMENT REGARDING FEDERAL FUNDING

This invention was made with government support under grant numbers HL084200 and GM103532 from the National Institutes of Health. The government has certain rights in the invention.

### BACKGROUND OF THE DISCLOSURE

Pulmonary infection is one of the leading causes of death and disability in the US and worldwide and accounts for greater than $40 billion/year in direct and associated costs in the U.S. alone. Despite initial gains in the late 19^{th} through mid-20^{th} centuries, recent efforts to further reduce the morbidity and mortality associated with bacterial and viral pneumonia have been largely ineffective. Furthermore, recent epidemics, such as the recent pandemic (pH1N1) influenza outbreaks, have highlighted the continued threat of emerging pathogens for which there are few if any effective treatments. Epidemiological studies suggest that in addition to pathogen prevalence and virulence, host factors play a critical role in determining both susceptibility to and outcome from pulmonary infections. Yet, our understanding of these factors remains limited, and there is a pressing need to identify therapeutic approaches that restore host defense in these populations.

### SUMMARY OF THE DISCLOSURE

The present disclosure is based on our observations that obesity-associated hyperleptinemia and not body mass per se drives the observed attenuation of the pulmonary inflammatory response, and that this effect could also impair the host response to pneumonia.

In the present disclosure, we examined circulating leptin levels and risk, severity, and outcome of pneumonia in two patient cohorts (The National Health and Nutrition Examination Survey (NHANES) and the ARDSNet-ALVEOLI and in mouse models of diet-induced obesity and lean hyperleptinemia.

We found that plasma leptin levels in ambulatory subjects (NHANES cohort) correlated positively with annual risk of respiratory infection independent of BMI. Subjects with Acute Respiratory Distress Syndrome (ARDS) (ALVEOLI cohort) and high circulating leptin levels were most likely to have developed ARDS from pneumonia, and in patients with ARDS and pneumonia, elevated leptin levels were strongly associated with 90 d mortality, independent of BMI. Furthermore, leptin levels were inversely correlated with plasma levels of IL-6, suggesting a suppressive effect. In obese mouse models of Klebsiella penumonia, pneumonia severity was positively correlated with both body weight and plasma leptin levels with a significant interaction term in multivariate analyses. In the less variable model of LPS-induced lung injury in obese mice, leptin levels and weight were both found to be inversely related to BAL neutrophilia in univariate analyses, yet only leptin levels remained significantly associated with this in multivariate analyses while weight did not. Furthermore, plasma G-CSF and IL-6 levels were found to be inversely related to leptin levels in this model, while there was no significant association between these variables and weight. We confirmed these associations in lean mice with induced hyperleptinemia, showing that both neutrophil recruitment to the lung following LPS injury and the response to bacterial infection were impaired in isolated hyperleptinemia. In vitro examinations of neutrophils from induced hyperleptinemia mice revealed that these cells have impaired chemotaxis response and accelerated apoptosis. In studies of neutrophil signaling to leptin, we found that murine neutrophils express ObRa mRNA but not ObRb, and neutrophils stimulated with leptin in vitro show increases in p-P38 and p-GSK3β (both products of the ObRa pathway), but not p-STAT3 (which requires the ObRb receptor isoform). These findings suggest that the association between obesity and elevated risk of pulmonary infection may be driven by hyperleptinemia, and that this association may be related to hyperleptinemia's effect on neutrophil response via ObRa. Thus, we believe hyperleptinemia augments the risk for and severity of both bacterial and viral pneumonias through its detrimental effects on neutrophil function, which include impairment of neutrophil chemotaxis and survival.

In one aspect, the present disclosure provides a method for determining whether an individual is at risk of developing pulmonary infections by measuring circulating leptin levels. The method comprises determining circulating leptin levels, comparing the levels to a control level and based on the comparison, identifying the individual to be as risk or not at risk of developing pulmonary infection.

The present disclosure also provides a method for reducing the risk for or reducing the severity of pulmonary infections in patients with elevated serum leptin levels comprising administration of one or more agents that suppress the levels of leptin or interfere with its actions. Examples of such patients include obese, pregnant, and dialysis patients, diabetics, and the aged.

### BRIEF DESCRIPTION OF THE FIGURES

FIG. 1: Pulmonary bacterial clearance is impaired in hyperleptinemic mice. Lean C57 Bl/6 mice were injected with 40 ug/kg recombinant leptin IP/d×14 d in a model of chronic hyperleptinemia (HL). HL and control mice (A) were oropharyngeally instilled with K. pneumoniae (2000 CFU) and whole lung CFU were determined 48 h later. Similar infections were performed in mice with diet-induced obesity and lean controls (60% vs 10% fat diet×20 wks; pre-infection weights: 31.8+/−1.0 g vs. 47.9+/−0.9 g; p<0.001) (B). n=8 mice/grp, *p<0.05; **p<0.01.

FIG. 2: Hyperleptinemic mice have decreased survival following H1N1 infection. HL and control mice were nasally infected with a sub-lethal dose of A/California/7/2009 H1N1 (3×10³ EIU) and followed for 15 d. Leptin given daily for 14 d prior to infection and at d+3. n=10 mice/group, *p=0.028 by Log-rank test.

FIG. 3: Pneumonia severity as measured by bacterial burden correlates with plasma leptin levels in K. pneumoniae-infected lean and obese mice. (A) Lung bacterial colony forming units (CFU) were determined at 48 h after K. pneumoniae infection in lean (10% fat diet) and diet-induced obese (60% fat diet) mice. In addition, (B) lung CFU was compared with mouse weight and (C) plasma leptin levels by linear regression. n=11 (10% diet) and n=9 (60% diet) animals. Dashed lines indicate 95% confidence intervals. *** P≦0.001 as determined by an unpaired Student's t-test (two-tailed).

FIG. 4: Plasma leptin levels inversely correlate with airspace neutrophilia in LPS-injured lean and obese mice. Previously we have reported that BAL neutrophil counts and mouse body weight are inversely associated (r²=0.32, P=0.02) following LPS-induced lung injury in diet-induced obese mice. We now compared BAL neutrophil counts and plasma leptin levels at 24 h after LPS-induced lung injury in diet-induced obese (60% fat diet, n=6) and lean (10% fat diet, n=5) mice by linear regression. Dashed lines indicate 95% confidence interval.

FIG. 5: Plasma leptin levels are inversely associated with plasma neutrophilic cytokine levels following LPS-induced lung injury in lean and obese mice. Plasma IL-6 and G-CSF levels were compared with either (A, B) plasma leptin levels or (C, D) body weight at 24 h after LPS-induced lung injury in diet-induced obese (60% fat diet) and lean (10% fat diet) mice by linear regression. Dashed lines indicate 95% confidence intervals.

FIG. 6: LPS-induced lung injury is attenuated in a mouse model of isolated hyperleptinemia. BAL neutrophil counts (n=6 (control) and n=8 (HL)) (A) were determined in i.p. leptin treated (14 days) C57Bl/6 WT mice compared to i.p. PBS treated controls at 24 h after LPS-induced lung injury and compared to plasma leptin levels by linear regression (n=6 (control) and n=8 (HL)) (B). Dashed lines indicate 95% confidence interval. ** P≦0.01 as determined by an unpaired Student's t-test (two-tailed).

FIG. 7: Neutrophil chemotaxis and survival are impaired in cells isolated from hyperleptinemic mice. Mature bone marrow neutrophils were isolated from uninjured HL and control mice and examined in vitro using (A) modified a Boyden chemotaxis chamber and (B) 12 h culture with Fas ligand (200 ng/mL)+/−G-CSF (25 ng/mL). *p=0.01

FIG. 8: Neutrophils express only ObRa mRNA and do not activate STAT3 with leptin exposure. Mature bone marrow neutrophils were isolated from wild type mice and examined using (A) RT-PCR of isolated mRNA with brain as a positive control, and (B) Immunoblotting of whole cell lysates for phospho-STAT3 after exposure to leptin vs. IL-6.

FIG. 9: Neutrophils phosphorylate p38 and GSK3 in response to leptin. Mature bone marrow neutrophils were isolated from wild type mice and examined using immunoblotting of whole cell lysates for phospho-p38 and GSKα/β after in vitro exposure to IL-6 (20 ng/mL), leptin (25 ng/mL) or G-CSF (25 ng/mL). GSK image has been altered to fit.

FIG. 10: Hyperleptinemia impairs neutrophil signaling response to G-CSF and IL-6. Mature bone marrow neutrophils were isolated from HL and control mice and examined using immunoblotting of whole cell lysates for phospho-STAT3 after in vitro exposure to (A) G-CSF (25 ng/mL) or (B) IL-6 (20 ng/mL).

FIG. 11: Neutrophils from obese mice without hyperleptinemia have normal p38 and STAT3 responses. Mature bone marrow neutrophils were isolated from ob/ob mice (weighing ˜45 g) and heterozygous littermates (weighing ˜25 g) and examined using immunoblotting of whole cell lysates for phospho-p38 and STAT3 after in vitro exposure to (A) LPS (100 ng/mL) or (B) G-CSF (25 ng/mL).

FIG. 12: BAL cytokines tend to be lower in hyperleptinemic mice following LPS exposure. BAL cytokine levels were determined at 24 h after LPS exposure in mice that received PBS or leptin (2 μg) i.p. injections for 14 days. n=11 per group. ** P≦0.01 as determined by an unpaired Student's t-test (two-tailed).

### DETAILED DESCRIPTION OF THE DISCLOSURE

Leptin was first discovered in the obese mouse as a serum factor that decreased food intake and body weight. Because of these initial observations, much of the earlier therapeutic attempt using this hormone has been in the treatment of obesity.

The present disclosure is based on the unexpected observations that increased circulating leptin levels may increase the risk of respiratory infections (such as pulmonary infections). In one aspect, this disclosure provides a method of determining whether an individual is at risk for developing pulmonary infection comprising obtaining a sample (test sample) of a biological fluid from the individual, and measuring the level of leptin in the test sample, comparing the level of leptin in the test sample to a control level of leptin, and diagnosing the individual as being as risk of developing pulmonary infection if the level of leptin in the test sample is greater than the control level. In one embodiment, the leptin levels are measured by immunological techniques. In one embodiment, the immunological technique is ELISA.

In one aspect, this disclosure provides a method of determining whether a population of individuals is at risk of developing respiratory infections (such as pulmonary infections) comprising obtaining biological fluid samples from a portion (or all) of the population, determining leptin levels in the samples, comparing the leptin levels in the population (or a portion of the population) to normal controls, and if the leptin levels in the population are greater than the normal control, identifying the population to be at risk of developing respiratory infection (such as pulmonary infection). If a determination is made that the population is at risk, then prophylactic measures may be instituted—such as, for example, prophylactic antibiotics and/or immunizations. In addition to a lack of respiratory infection, the normal controls may be matched for age, gender, and other attributes.

In one embodiment, the disclosure provides a method for reducing the risk for developing pulmonary infections, and/or augmenting the therapy thereof, comprising administration or one or more agents that will reduce leptin levels or will interfere with leptin function. Pulmonary infections include but are not be limited to pneumonia, upper respiratory infection, bronchitis, bronchiolitis, or bronchiectasis from bacterial, viral, or other infectious pathogen, flares of COPD and asthma, or other underlying lung diseases. In one embodiment, the disclosure provides a method for reducing the risk for developing pulmonary infections, and/or augmenting the therapy thereof, comprising administration or one or more agents that will reduce leptin levels or will interfere with leptin function (referred to herein as leptin action antagonists). In one embodiment, the leptin action antagonists may be antibodies or small molecules against leptin (including nanobodies—see Zabeau et al., Biochem J. (2012), 441:425-434), antagonists of leptin (such as superactive mouse leptin antagonist (SMLA—see Shipilman et al., J. Biol Chem 286:4429-4442, 2011, Solomon et al., Am. J. Physiol Endocrinol Metab 306: E14-E27, 2014). The SMLA may be PEGylated (PEG-SMLA) and is commercially available from Protein Laboratories in Rehovot. In one embodiment, leptin function is inhibited by direct blockade of leptin-induced signaling inhibitors including inhibitors of PTEN (such as SF1670, commercially available from Cellagen Technology) and PTP1B (such as Trodusquemine, Ohr Pharmaceutical, 539741, Calbiochem). In one embodiment, leptin function/action is inhibited by pharmacologic inhibitors of JAK2 (such as AZD1480, Astra Zeneca, TG101348, Selleckchem), PI₃K (such as AZ8186, Astra Zeneca), GSK3β (TDZD), and Akt (such as AZD5363, Astra Zeneca). Other targets include IRS & 2, p85α, Grb2, p38, ERK1/2, and JNK.

In one embodiment, the leptin antagonist is a leptin nanobody (also known as single domain antibody). Nanobodies can be custom-made commercially—such as from Creative Biolabs, Shirley, N.Y. Leptin nanobodies can also be prepared by methods described in the literature—such as by Zabeau et al. (Biochem J. 2012, 441:425-434, McMurphy et al., 2014, PLOS One, 9(2):e89895)—the disclosures of these references are incorporated herein by reference.

The method comprises administering to an individual in need of treatment a therapeutically effective amount of a leptin action antagonist. The leptin action antagonist may reduce circulating leptin levels (as measured in blood, serum or plasma) or may inhibit the action of leptin. The individual in need of treatment may be an individual who has high leptin levels. For example, such individuals may include individuals who are obese, pregnant, and dialysis patients, diabetics, and the aged (such as over the age of 60, 65 or 70).

Leptin levels may be measured by any known method in the art. The leptin levels may be free leptin levels or total leptin levels. In one embodiment, the leptin levels are fasting levels (such as, for example, after 12 hours of fasting). In one embodiment, the levels are non-fasting levels (random levels). Studies on free and bound leptin and in lean and obese subjects can be found in Sinha et al., (J. Clin Invest. 1996, Sep. 15; 98(6):1277-82, incorporated herein by reference). Leptin levels may be measured by immunological methods such as enzyme linked immunosorgent assay (ELISA). In one embodiment, the levels are measured by contacting a fluid—e.g., biological fluid (such as whole blood, plasma or serum) or cell culture fluid (such as cell culture supernatant) with an antibody (or antigen binding fragment thereof) and then detecting the leptin-antibody (or leptin-antibody fragment) complex. Leptin ELISA kits are commercially available—such as from R&D Systems, or Life Technologies. In one embodiment, the assay involves detection and measurement of leptin antibody complex, wherein the antibody is immobilized to a substrate. The use of a second (labeled) antibody allows the detection of the leptin antibody complex. The intensity of the label is directly proportional to the level of leptin. Quantification can be carried out by comparison to standard curve.

Determining the appropriate dosage of leptin action antagonists is within the purview of clinicians. As a guidance, the dosage should be targeted to return the leptin levels to normal range (in therapies that reduce these) and/or the restoration of neutrophil function as demonstrated by normalization of their chemotaxis, survival, and signaling response to critical cytokines such as G-CSF. In one embodiment, the dosage is 0.1 to 10 mg/kg. In one embodiment, it is from 2.5 to 5 mg/kg. The dose may be administered multiple times a week (such as, for example, three times a week). It may be continued as necessary, for example from 1 to 4 weeks.

Pharmaceutical compositions of leptin action antagonists or antibodies may comprise effective amounts of the leptin antagonists and/or antibodies together with pharmaceutically acceptable diluents, preservatives, solubilizers, emulsifiers, adjuvants and/or carriers. Such compositions may include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used, and this may have the effect of promoting sustained duration in the circulation. Agents like oleic acid, linoleic acid and linolenic acid may be added to aid delivery. Further, the formulations may be prepared as controlled release formulations. See, e.g., Remington's Pharmaceutical Sciences, 18th Ed. (1990, Mack Publishing Co., Easton, Pa. 18042). The compositions may be prepared in liquid form, or may be in dried powder, such as lyophilized form, or as aerosolized form.

The formulations may be administered via injection (i.v., i.p., i.m. and the like), oral, pulmonary, nasal, transdermal or other forms of administration. In one embodiment, the leptin action antagonist formulation may be delivered directly to the respiratory system via a formulation that is suitable for inhalation. For example, the compositions of the invention may be used in the form of drops or sprays (e.g., a nasal spray, aerosol spray, or pump spray) or other vehicles for nasal administration (intranasal delivery). Aerosol spray preparations can be contained in a pressurized container with a suitable propellant such as a hydrocarbon propellant. Pump spray dispensers can dispense a metered dose or a dose having a specific particle or droplet size. Any dispensing device can be arranged to dispense only a single dose, or multiple doses. The formulations may be provided in the form of a dispersible dry powder which can be then prepared for delivery by inhalation. In one embodiment, it is continuously administered to a patient such as through intravenous route or other suitable routes.

The dosage and number of administrations are dependent on the severity of the condition as well as individual patients. Adjusting the dosage and frequency of administration is well within the purview of a skilled physician.

In one embodiment, a suitable dosage is a regimen such that the symptoms of pulmonary infection are reduced. The administration of the leptin action antagonists can be monitored clinically and administration reduced or stopped when the clinical manifestation of pulmonary infection are no longer detectable. Examples of individuals who can benefit from such approach include obese, elderly, pregnant, etc.

In one embodiment, circulating leptin levels can be used to predict risk of pulmonary infection in both an individual and in a population of individuals. In an embodiment, circulating leptin levels provide a useful screening technique in populations to determine who should be prophylaxed whether by leptin antagonists or other measures (e.g. vaccines, prophylactic antimicrobials in an outbreak, etc.). For determining if individuals have higher leptin levels, normal or control levels may be established. In one embodiment, the normal free (non-fasting) leptin levels are 18-22 ng/ml. In one embodiment, the level determination in an individual is carried out by the same method as the determination of the normal or control levels. In a study by Sinha et al., leptin levels are reported to be basal vs 24 h fasting: 19.6+/−1.9 vs. 1.3+/−0.4 ng/ml for lean individuals, and basal vs 24 h fasting: 28.3+/−9.8 vs. 14.7+/−5.3 ng/ml for obese individuals. Thus, in one embodiment, if an individual or individuals are found to have levels that are higher than the level in normal controls, the individual or individuals are considered to be at risk of developing a pulmonary infection. In one embodiment, if the individuals have leptin levels which are at least 10%, or 20% or more greater or have twice the levels in normal controls (when matched to respective fasting or random levels), the individual may be considered at risk of developing a pulmonary infection. In one embodiment, the leptin levels may be considered to be higher than normal if the fasting leptin levels are >10 ng/ml or if random leptin levels are >25 ng/ml. It is within the purview of those skilled in the art to determine if the test levels are higher than the control levels. The control levels may be matched for other independent risk factors—such as smoking, gender, alcoholism etc. In an embodiment, fasting leptin levels are compared to normal control fasting leptin levels. In an embodiment, non-fasting leptin levels can be compared to non-fasting normal control leptin levels.

In one embodiment, the leptin action antagonist administration can be accompanied by the administration of antimicrobial agents such as antibiotics or antifungal agents, and/or by immunizations. The leptin antagonist formulation can be administered before, during or after the administration of the antimicrobial agents or immunizations.

In one embodiment, the present disclosure provides a method for identifying a population at risk of pulmonary infections comprising determining circulating leptin levels in individuals from the population and if the levels are above a normal range, then identifying the population as being at risk. The method may further comprising the step of prophylactic treatment of individuals in the population at risk comprising administering antimicrobial agent to the individuals, or vaccinations. The antimicrobial agent may be antibacterial or an anti-viral agent.

In one embodiment, the disclosure provides a method of treating or preventing respiratory infection in an individual who has been exposed to a respiratory pathogen comprising administration to said individual a leptin action antagonist in a pharmaceutically acceptable carrier, wherein administration of said leptin action antagonist results in prevention of the respiratory infection or results in reducing the severity of infection.

The invention is further described through the following examples which are to be considered as illustrative and are not intended to be limiting.

### Example 1

This example describes that hyperleptinemia augments the risk for pulmonary infection in part through its detrimental effects on neutrophil function, which include impairment of neutrophil chemotaxis and survival.

Risk of respiratory infection increases with rising serum leptin levels in the general population—We used data from the National Health and Nutrition Examination Survey (NHANES III) to test the association of serum leptin levels with self-reported pulmonary infections in a representative sample of non-institutionalized adults (n=6,252). Patients were identified as having had respiratory infection (n=396) if they reported one or more pneumonias in the last year (n=134) or one or more URIs (flu or bronchitis; n=288) in the past three weeks. We used univariate logistic regression to assess the relationships between potential covariates and respiratory infection, and those that were associated with the outcome with p<0.1 were retained in a multivariate logistic model. In univariate logistic regression on subjects with complete data (n=30,818), age (p<0.0001), gender (p<0.0001), race (p=0.0003), and smoking status (p=0.0003) were found to correlate significantly with the annual risk of infection. Markers of dyslipidemia (LDL, HDL, and total cholesterol) were also examined and found not to be associated with risk (p=0.76).

Further analysis of a smaller subset of this cohort in which plasma leptin levels were measured (n=6,252) revealed a significant association between leptin and respiratory infection (OR=1.109 per ng/ml; 95% CI 1.003, 1.034; P=0.019) in univariate analyses (Table 1). In multivariate linear regression analysis including age, sex, BMI, social status indicators, smoking, diabetes and renal function, the annual risk for respiratory infection is only significantly correlated with serum leptin level, gender and smoking status. P≦0.05 is considered significant. Several metabolic variables were positively associated with leptin levels in this cohort, including BMI (p<0.0001), HbA1c (p<0.0001), and renal function (p<0.0001). These findings indicate that the elevated risk for respiratory infection seen in patients with obesity, diabetes, and/or renal failure are mediated by leptin.

TABLE 1

| | Odds | | |
|---|---|---|---|
| Respiratory infection (n = 396) | Ratio | 95% CI | P |
| Univariate Analysis | | | |
| Leptin (ng/mL) | 1.019 | [1.003, 1.034] | 0.019* |
| Age (years) | 1.003 | [0.994, 1.012] | 0.51 |
| Male | 0.622 | [0.433, 0.894] | 0.011* |
| White Race | 0.975 | [0.655, 1.453] | 0.90 |
| Education (0-17 years) | 0.987 | [0.935, 1.042] | 0.62 |
| Income<$20, 000 | 1.004 | [0.687, 1.465] | 0.99 |
| Married (or living as married) | 1.051 | [0.670, 1.647] | 0.83 |
| Body Mass Index (kg/m²) | 1.017 | [0.988, 1.048] | 0.25 |
| Current Smoker | 1.950 | [1.352, 2.812] | 0.0006* |
| Diabetes Mellitus | 1.383 | [0.788, 2.429] | 0.25 |
| Glycated Hgb A1C (%) | 1.116 | [0.963, 1.293] | 0.14 |
| Creatinine clearance | 1.004 | [0.995, 1.013] | 0.34 |
| (ml/min/1.74 m²) | | | |
| Multivariate Analysis | | | |
| Leptin (ng/mL) | 1.016 | [1.000, 1.032] | 0.049 |
| Male | 0.692 | [0.481, 0.995] | 0.047 |
| Current Smoker | 2.139 | [1.482, 3.086] | 0.0001 |

We also examined the incidence of urinary tract infections in the cohort in which leptin levels were measured. Although the risk for urinary tract infection was associated with serum leptin levels in univariate analysis, it was not in multivariate analysis, suggesting that high leptin levels may be associated specifically with respiratory infection but not other infections.

Risk of death from severe pneumonia increases with rising blood leptin levels—We examined patients with severe pneumonia (n=148) who developed respiratory failure and ARDS and were enrolled in the NIH ARDS Clinical Trials Network ALVEOLI study. Banked plasma samples from the day of subject enrollment were assayed by leptin ELISA, and the association between log transformed leptin levels and subsequent death within 90 d was analyzed by logistic regression adjusting for BMI, APACHE, gender, and diabetic status. Elevated plasma leptin was associated with increased risk of death in patients with pneumonia (OR 1.31, 95% CI 1.02-1.70, p=0.037), but was not associated with death in patients with other risk factors for ARDS in the trial (p=0.81). Furthermore, elevated leptin levels at enrollment in the trial were found to correlate with the diagnosis of pneumonia as the identified risk factor for developing ARDS in the cohort as a whole (OR=0.616; 95% CI=0.0875, 1.1447; P=0.022; n=385). These findings suggest that hyperleptinemia is not only associated with the risk of pulmonary infection, but also death in severe cases of pulmonary infection.

Induced hyperleptinemia impairs pulmonary bacterial clearance in mice—To determine if chronic hyperleptinemia in the absence of obesity or other hyperleptinemia-associated condition might itself impair the response to pulmonary infection, we examined a sub-lethal Klebsiella pneumoniae model in lean mice with experimentally-induced hyperleptinemia. We developed a protocol of repeated daily IP recombinant leptin injection that yielded sustained hyperleptinemia (HL) compared to IP PBS controls (22.6±3.3 vs. 1.3±0.7 ng/mL; p<0.0001), though lower than levels seen in mice with a relevant hyperleptinemic condition such as diet-induced obesity (38.2±4.8 ng/mL). In this protocol, leptin-treated mice had no weight loss (d14 weights: HL 19.2±0.2 vs. control 18.7±0.5 g; p=0.29), hyperglycemia (100.2±4.3 vs. 108.2±3.5 mg/dL; p=0.19), dyslipidemia or plasma inflammatory cytokine elevation (data not shown). However, following infection, hyperleptinemic (HL) mice demonstrated a significant defect in bacterial clearance compared to controls (FIG. 1A), that was comparable to that seen in mice with diet-induced obesity (FIG. 1B). Further, by regression analysis, no association between bacterial burden and mouse weight was observed. Thus, hyperleptinemia itself appears to impair the response to bacterial pneumonia and may underlie the increased susceptibility seen in obesity

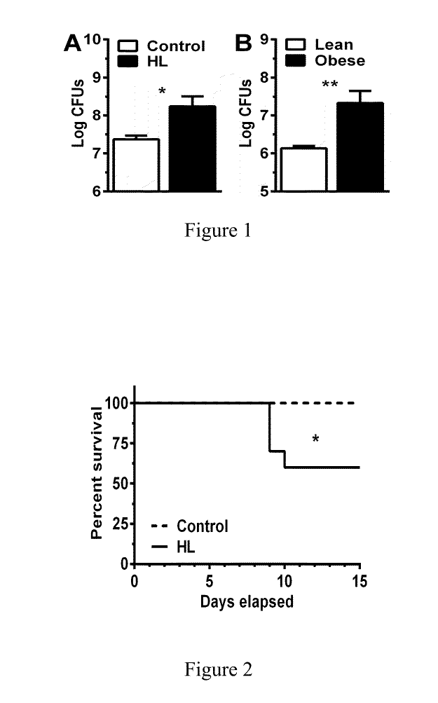

Mortality from pH1N1 influenza is increased in hyperleptinemic mice—We next examined whether non-obese hyperleptinemia would confer similar susceptibility to pH1N1 infection using a murine-adapted virus (A/California/7/2009 H1N1). Although all control mice survived infection, 40% of leptin-treated mice died by d+10 of infection (FIG. 2), implicating the effects of hyperleptinemia in susceptibility to influenza.

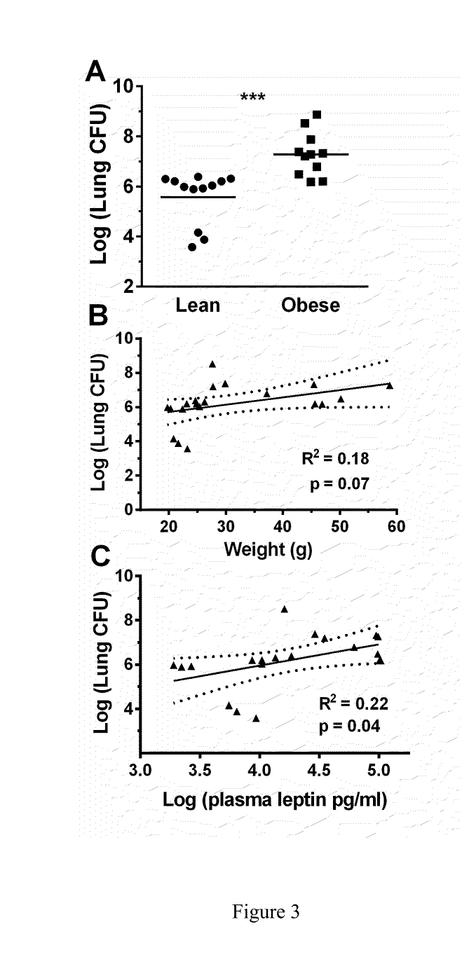

Plasma leptin levels and not body weight correlate with bacterial burden in a mouse model of obesity. As obesity is the most prevalent cause of hyperleptinemia, we next investigated whether the observed associations between hyperleptinemia and pneumonia risk and severity could be replicated in animal models of obese pneumonia. We examined Klebsiella pneumoniae infection in a diet-induced obesity (DIO) mouse model to mimic human obesity and pneumonia. Although pneumonia severity, as measured by whole lung bacterial colony-forming units (CFU) at 48 h, was increased in the obese mice as a group (FIG. 3A), there was no association between bacterial burden and mouse weight in regression analysis (FIG. 3B). However, a positive association was found with plasma leptin levels (FIG. 3C).

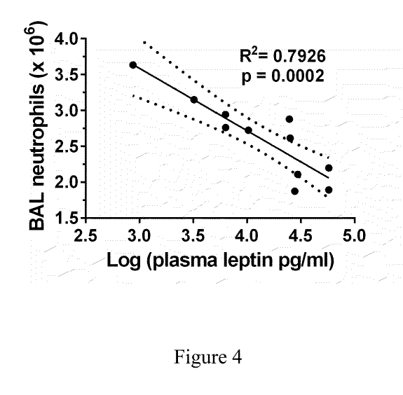

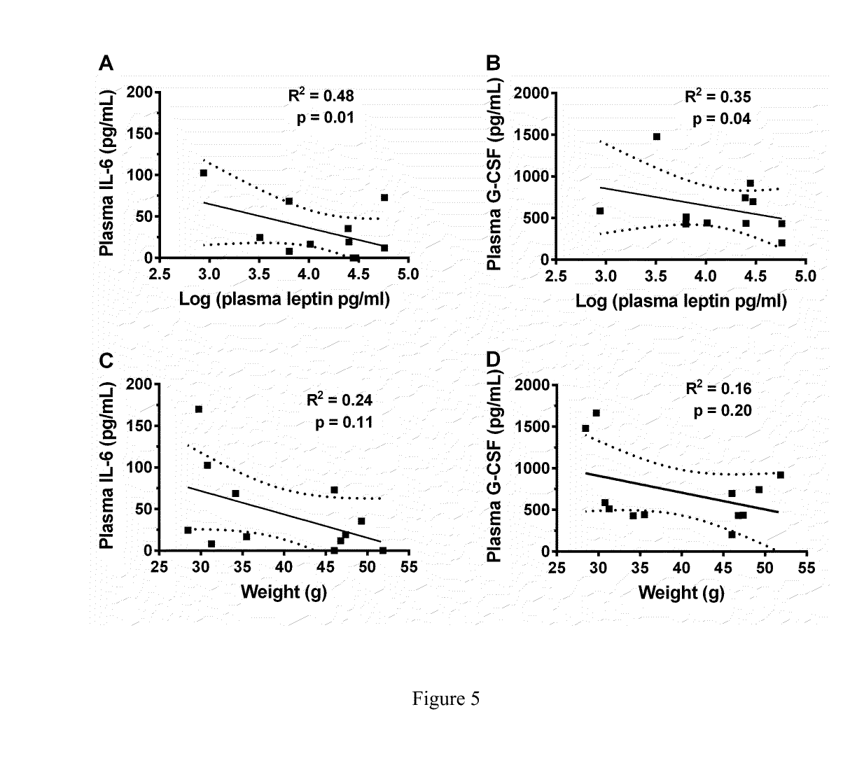

Plasma leptin levels are inversely associated with airspace neutrophilia in obese mouse models of lung injury—Given our new findings associating circulating leptin levels with pulmonary infection, we examined the association between plasma leptin and airspace neutrophilia in these mouse models. In diet-induced obese (DIO) and lean mice, hyperleptinemia was strongly associated with attenuated BAL neutrophilia (FIG. 4) and in multivariate analysis, leptin levels remained significantly associated with BAL neutrophilia (p=0.021), while body weight did not (p=0.80). Furthermore, an inverse association was found between plasma leptin levels and plasma IL-6 levels (p=0.01) (FIG. 5A). Given the changes observed in airspace neutrophil counts in this mouse model following injury, we also examined the neutrophilic cytokine G-CSF, and found it to be inversely associated with plasma leptin levels (p=0.04) (FIG. 5B). However, no association was found between body weight and IL-6 or G-CSF in this model (FIGS. 5C and 5D). These results suggest that obesity-associated hyperleptinemia underlies the previously established associations between obesity and impaired cytokine response following lung injury. These findings suggest that the defects in neutrophil recruitment we have previously related to obesity may instead be related to obesity-associated hyperleptinemia.

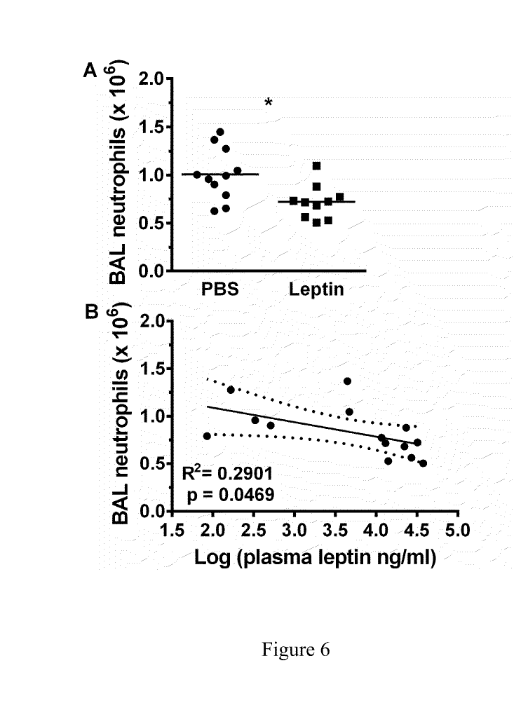

Hyperleptinemia impairs the development of pulmonary neutrophilia—To determine if hyperleptinemia in the absence of obesity and other elements of the metabolic syndrome might itself impair neutrophil response to the lung, we exposed HL mice to nebulized LPS exposure to examine the immune response. Levels of blood neutrophilia were comparable between HL and control mice, but airspace neutrophil accumulation was attenuated in HL mice (FIG. 6A), and linear regression analysis showed a significant inverse correlation between BAL neutrophil counts and plasma leptin levels in these lung-injured mice (FIG. 6B). In addition, BAL IL-6 levels were significantly decreased in the hyperleptinemic mice (FIG. 12). We next examined cytokine response in alveolar macrophages isolated from uninjured HL mice. These cells were hyperresponsive to LPS compared to control (KC 3551±132 vs. 2494±80 pg/mL, IL-6 899±29 vs. 699±44, MCP-1 173±10 vs. 125±1, TNFα 5173±107 vs. 2622±433, all p<0.01), suggesting that the attenuation of LPS-induced airspace neutrophilia in hyperleptinemic mice reflects primary defects in neutrophil function.

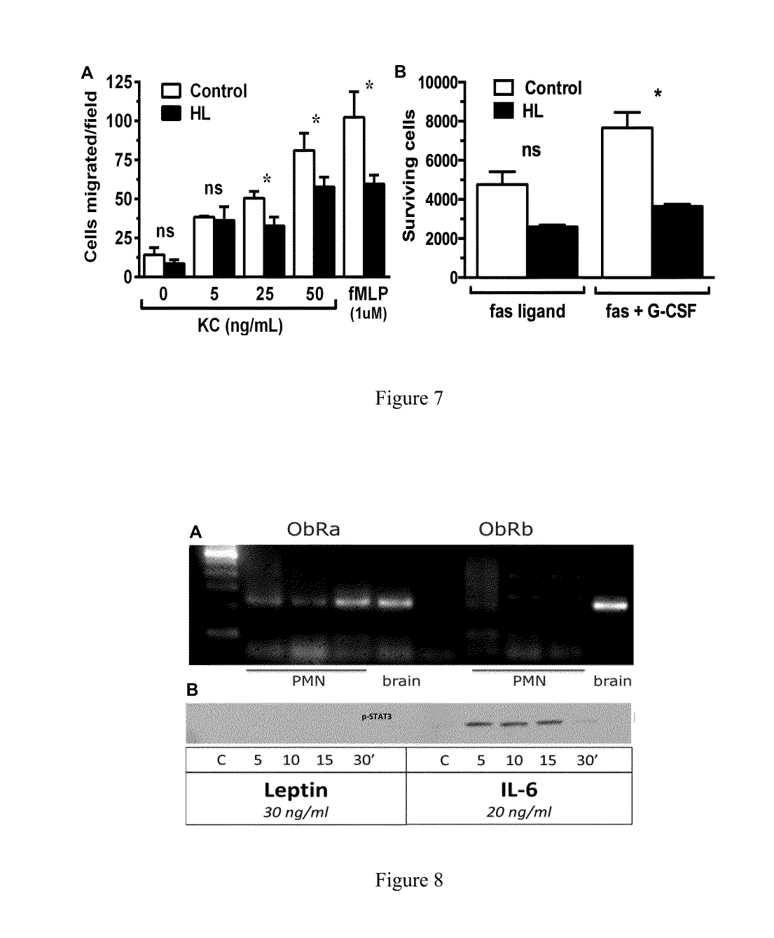

Hyperleptinemia impairs neutrophil chemotaxis and survival—We next examined hyperleptinemia's effects on neutrophil chemotaxis and survival, two functions that are critical to the development and persistence of airspace neutrophilia. Chemokine and bacterial peptide-induced in vitro chemotaxis (FIG. 7A) were impaired in neutrophils isolated from HL compared to control mice. Neutrophil survival following Fas ligand exposure was also impaired, and could not be rescued by G-CSF (a powerful neutrophil anti-apoptotic) (FIG. 7B). The roles these and other potential defects in neutrophil function may play in the hyperleptinemia-driven susceptibility to bacterial and viral pneumonia remain unknown.

### Methods

The National Health and Nutrition Examination Survey (NHANES III) database was used to test the association of serum leptin levels to self-reported infections in a representative sample of non-institutionalized adults. NHANES is an ongoing data collection of the US Centers for Disease Control (webpage: cdc.gov/nchs/nhanes.html). Each sample is comprised of subjects randomly selected form the US population using a stratified sampling scheme. Consenting subjects submit to an extensive interview, examination and collection of blood (Centers for Disease Control and Prevention).

Eligible subjects included adults aged 18 years or older at the time of the interview that provided data for all the outcomes and potential confounders and had leptin measures reported. Upper Respiratory Infection (URI) was coded as the answer to “In the past three weeks have you had any respiratory infections, such as flu, pneumonia, bronchitis, or a severe cold?” Pneumonia was coded as the answer to “During the past 12 months, have you had . . . Pneumonia?” The primary outcome measure Respiratory Infection, was created by combining the responses to URI and Pneumonia. Subjects who reported one or more pneumonias in the last year, or one or more URIs in the past three weeks, or both, were coded as positive.

ALVEOLI study: Patients with ARDS in our study participated in an RCT of lower versus higher positive end-respiratory pressure, and these patients also received 6 cc/kg tidal volume. Briefly, patients were eligible if they required mechanical ventilation and met criteria for acute lung injury according to the American-European Consensus Conference (AECC) definition (Bernard et al., Am J. Respir Crit care Med 149:818-824, 1994). Patients with body weight greater than one kilogram per centimeter of height were excluded. For each participant, Acute Physiology and Chronic Health Evaluation (APACHE) score during the 24 hours following ICU admission was calculated, and the physician investigator identified the primary risk factor for the development of ALI (sepsis, trauma, pneumonia, aspiration, multiple transfusions, or other) (Eisner et al., Am J. Respir Crit care Med 164:231-236).

In the NHANES study human serum leptin levels (after an overnight fast) were determined by radioimmunoassay with a polyclonal antibody raised in rabbits against highly purified recombinant leptin (US Department of Health and Human Services (DHHS) National Center for Health Statistics 2001). Serum IL-6 and leptin levels in samples from the ALVEOLI study were determined by ELISA (both Quantikine, R&D systems, Minneapolis, Minn.) according to manufacturer's protocol.

For animal studies, eight to twelve week old female C57Bl/6 mice (Harlan, Indianapolis, Ind.) were fed high vs. normal fat chow (60% vs. 10% fat; Research Diets, New Brunswick, N.J.) for 20 weeks or intraperitoneally (i.p.) injected with PEGylated leptin (2 μg in 200 μl PBS) or PBS control (200 μl) daily for 14 days. Mouse weights and food intake were monitored and daily and average food intake was calculated per mouse. Animals were housed in the animal facilities at the University of Vermont and all experimental animal procedures were approved by the University of Vermont Institutional Animal Care and Use Committee.

For murine exposures, murine Klebsiella pneumoniae (43816 serotype 2, ATCC, 2×10³ CFU) infections were performed by oropharyngeal (o.p.) aspiration. Lipopolysaccharide lung injury was induced by nebulized LPS (E. coli 0111:B4, Sigma, St. Louis, Mo.) (Kardonowy et al., 2012, Am. J. Respir Cell Mol Biol., 47:120-127).

For murine lung analysis, airspace lavage cell counts and cytokine levels, as well as bacterial CFU where appropriate were determined at 48 h (K. pneumoniae) and 24 h (LPS) after exposure.

For murine cytokine analysis, murine IL-10, IL-6, KC, MCP-1, TNFα and G-CSF levels in plasma and BAL were assessed using a Bio-Plex suspension array system (Bio-Rad, Hercules, Calif.). Plasma and BAL leptin concentrations were measured by ELISA (mouse leptin Quantikine, R&D Systems) according to manufacturer's protocol.

Fasting blood glucose levels were determined after an overnight fast by a glucometer (Nipro diagnostics, Fort Lauerdale, Fla.). In addition, plasma LDL and cholesterol levels were assayed using an Advia Chemistry System (Siemens, Tarrytown, N.Y.).

Statistical analysis: Analyses of the data derived from the NHANES III database were performed by univariate linear regression in order to confirm that each of the potential confounders was associated with the independent variable (serum leptin level). Thereafter, univariate logistic regression analysis was used to assess the relationships between the potential confounders and respiratory infection. Those that were associated with both the predictor and the outcome with P<0.1 were retained in an adjusted multivariate logistic model. The adjusted odds ratio (OR), 95% confidence interval (CI) and P-value was calculated for each retained variable and accepted a P-value <0.05 on the OR for leptin as evidence of statistical significance. NHANES analyses were performed with SAS software (Cary, N.C.).

When analyzing ARDSNet data, leptin data were natural log-transformed due to non-normal distribution. Univariate analysis were performed with linear or logistic regression for continuous and categorical variables, respectively. Multivariable linear and logistic regressions with robust standard errors were used to evaluate the associations between leptin and our dependent variable of interest (IL-6) with adjustment for confounders. In our multivariable models, BMI and APACHE score were fit as a linear continuous variable. Inflammatory biomarker levels were natural log-transformed due to non-normality. Gender and comorbid diabetes were dichotomous, and risk factor for ALI was fit as an indicator variable. All analyses were performed with Stata 9.0 or greater (College Station, Tex.). Statistical significance was defined as a two-sided P-value ≦0.05.

Murine data were represented as mean, and analysis of differences between experimental groups was performed by Student t test. Differences between mouse weights and food intake over time between two groups were analyzed by repeated measures ANOVA. Correlations between murine plasma leptin levels or weight and lung CFU levels, BAL neutrophil counts and plasma cytokine levels, were analyzed by linear regression. All analyses were performed using Prism 6 software (GraphPad). Results with P≦0.05 were considered statistically significant.

### Example 2

This example describes that hyperleptinemia impairs neutrophil function through persistent ObRa-mediated stimulation of the JAK/IRS/PI3K pathway leading to downstream inhibition of both cytokine and TLR-induced responses.

Neutrophils express ObRa but not ObRb—We examined mRNA isolated from wild type mouse bone marrow neutrophils and brain (positive control) using RT-PCR and primers for both short (ObRa) and long (ObRb) forms of the leptin receptor. Although ObRa mRNA is present in neutrophils (FIG. 8A), we could find no evidence of ObRb in these cells. We next performed immunoblots (IB) for phospho-STAT3 in neutrophils exposed to rleptin or IL-6 (positive control). Leptin did not induce neutrophil STAT3 activation (FIG. 8B), the hallmark of ObRb signaling, confirming that ObRb is not expressed in these cells at physiologically relevant levels.

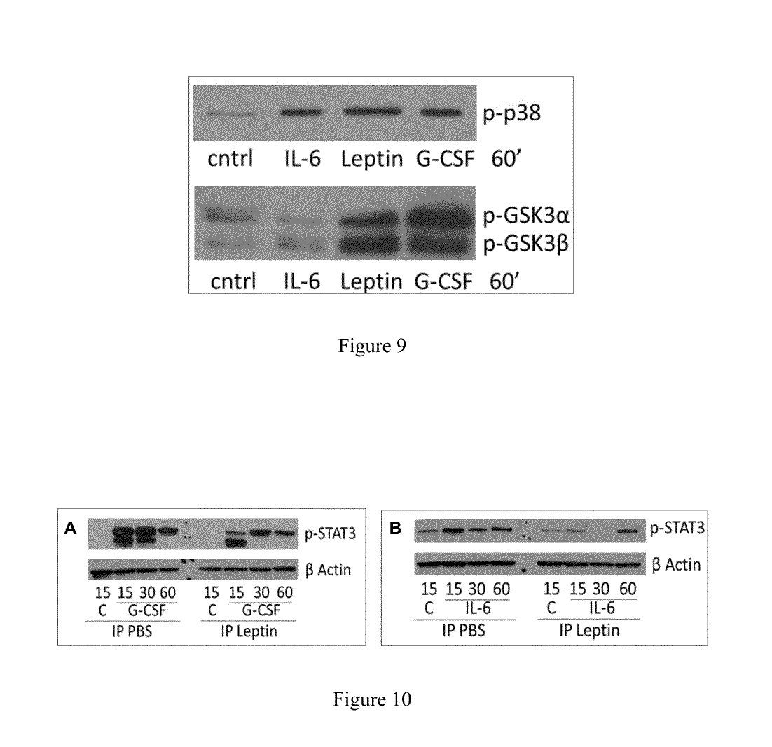

Leptin induces neutrophil p38 activation and GSK3 inhibition—To determine whether leptin signaling in neutrophils could be detected in downstream targets of the ObRa/JAK2 pathways, we examined cells stimulated with IL-6, leptin, or G-CSF. Under normal conditions, leptin induces both MAPK (p38) and GSK3α/β phosphorylation in neutrophils (FIG. 9), suggesting signal transduction through both Grb2 and IRS/PI3K/Akt pathways.

Hyperleptinemia impairs neutrophil signaling—We next examined the effects of sustained hyperleptinemia on neutrophil signaling using neutrophils isolated from uninjured HL and control mice. Following exposure to G-CSF, IL-6, or LPS, STAT3 and p38 activation was impaired in neutrophils from HL mice (FIG. 10). These findings suggest that sustained HL in vivo leads to suppression of TLR4 signaling proximal to p38 (possibly at the adapter protein level), and of STAT3, likely at the level of JAK/STAT interaction. These findings are consistent with inhibition of the known targets of GSK3β, PTEN, and PTP1B.

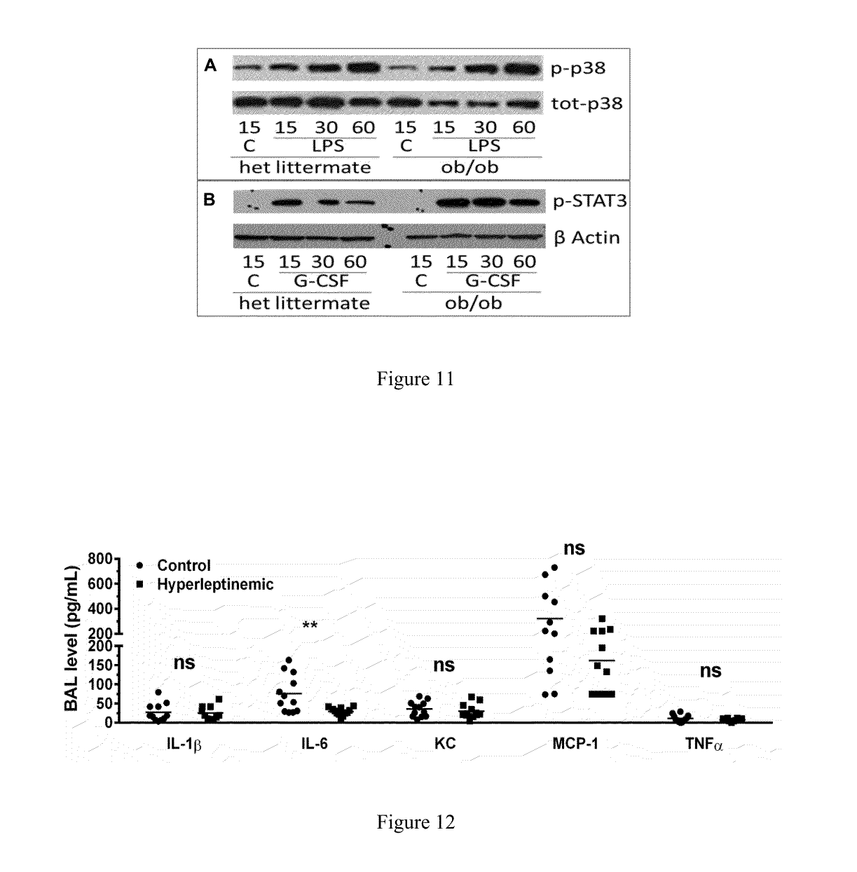

Obesity in the absence of hyperleptinemia does not impair neutrophil signaling response to LPS or G-CSF—We next examined obese aleptinemic (ob/ob) mice to preliminarily confirm that the effects of obesity on neutrophil signaling are distinct from those attributable to hyperleptinemia using neutrophils from uninjured ob/ob and heterozygous control mice. Following exposure to G-CSF or LPS in vitro, STAT3 and p38 activation normal to exaggerated in neutrophils from ob/ob mice (FIG. 11). These findings further suggest that HL effects are dissociable from obesity.

While the invention has been described through specific embodiments, those skilled in the art will recognize that routine modifications to the disclosure can be made and such modifications are intend to be within the scope of this disclosure.

1. A method of determining whether an individual is at risk for developing pulmonary infection comprising obtaining a sample of a biological fluid from the individual, and measuring by immunological techniques the level of leptin in the test sample, comparing the level of leptin in the test sample to a normal control level of leptin, and diagnosing the individual as being as risk of developing pulmonary infection if the level of leptin in the test sample is greater than the normal control level.

2. The method of claim 1, wherein the immunological technique is enzyme linked immunosorbent assay.

3. The method of claim 1, wherein the biological fluid is blood, plasma or serum.

4. The method of claim 1, wherein the leptin levels are fasting leptin levels.

5. A method of determining whether a population of individuals is at risk of developing pulmonary infection comprising obtaining samples of biological fluids from a portion of said population, measuring by immunological techniques the level of leptin in the samples, comparing the levels of leptin in the samples to a normal control, and identifying the population to be at risk of pulmonary infection if the level of leptin in the sample of the population is higher than the normal control level.

6. The method of claim 5, wherein the immunological technique is enzyme linked immunosorbent assay.

7. The method of claim 5, wherein the biological fluid is blood, plasma or serum.

8. The method of claim 5, wherein the leptin levels are fasting leptin levels.

9. A method of reducing the severity of pulmonary infection comprising administering to an individual in need of treatment, an effective dose of leptin action antagonist in a pharmaceutically acceptable carrier, wherein said administration reduces the severity of the infection.

10. The method of claim 9, wherein the infection is caused by an infectious agent including bacteria or virus.

11. The method of claim 9, wherein leptin action antagonist is administered by a route selected from the group consisting of: injection, oral, pulmonary, nasal, and transdermal.

12. The method of claim 9, wherein leptin actin antagonist is administered by inhalation.

13. The method of claim 9, wherein the individual is also administered an antimicrobial agent prior to, during, or after administration of the leptin function antagonist.

14. The method of claim 9, wherein the antimicrobial agent is an anti-bacterial or an anti-viral agent.

15. The method of claim 9, wherein the leptin action antagonist is a nanobody.
